(19) United States
(12) Patent Application Publication
(10) Pub. No.: US 2026/0331247 A1
(43) Pub. Date: Oct. 22, 2026

(54) ELECTRONIC DEVICE FOR ASSISTING TREATMENT FOR SUBJECT

(71) Applicant: CANON KABUSHIKI KAISHA, Tokyo (JP)

(72) Inventor: KAZUKI MIYAMOTO, Tokyo (JP)

(21) Appl. No.: 19/642,102

(22) Filed: Apr. 8, 2026

(30) Foreign Application Priority Data
Apr. 16, 2025 (JP) 2025-067649

(51) Int. Cl. G16H 20/00; G16H 30/40; G16H 50/20

(52) U.S. Cl. CPC G16H 20/00; G16H 30/40; G16H 50/20

(57) ABSTRACT

An electronic device according to the present disclosure includes one or more processors and memory configured to an image acquisition process of acquiring a captured image, an information acquisition process of acquiring, from the captured image, physique information regarding a physique of a subject of treatment, and a control process of performing control to provide a user who provides the treatment for the subject, with a notification for assisting the treatment, on a basis of the physique information.

### BACKGROUND

### FIELD OF THE TECHNOLOGY

The present disclosure relates to an electronic device for assisting a treatment for a subject.

### DESCRIPTION OF THE RELATED ART

A cross reality (XR) technology using an information processing apparatus has attracted attention, a typical one of which is a head-mounted display (HMD). The term XR is a generic term for technologies including virtual reality (VR), augmented reality (AR), and mixed reality (MR). The XR technology is expected to be utilized in various fields such as study of product design, improvement of a manufacturing process in a factory, and simulation in building designing. In particular, there is growing expectation for utilization of AR technology and MR technology in the field of medicine.

Meanwhile, one of problems in the field of medicine is a difficulty in providing appropriate lifesaving treatment for sudden cardiac arrest (SCA). International resuscitation guidelines recommend performing cardiopulmonary resuscitation (CPR) on an injured or diseased person suffering SCA as soon as possible. However, it is difficult for a person who has not been adequately trained professionally for CPR to take an appropriate lifesaving measure for an injured or diseased person suffering SCA when faced with such an emergency situation.

Japanese Patent Laid-Open No. 2017-209556 discloses a technology of informing the rescuer of information for assisting the lifesaving treatment.

Appropriate treatment varies depending on the injured or diseased person. However, the technology disclosed in Japanese Patent Laid-Open No. 2017-209556 provides information to the rescuer according to a certain criterion, which may prevent an appropriate treatment from being provided.

### SUMMARY

The present disclosure provides a technique that enables a user who provides a treatment to provide an appropriate treatment regardless of a subject of the treatment.

An electronic device according to the present disclosure includes one or more processors and memory configured to an image acquisition process of acquiring a captured image, an information acquisition process of acquiring, from the captured image, physique information regarding a physique of a subject of treatment, and a control process of performing control to provide a user who provides the treatment for the subject, with a notification for assisting the treatment, on a basis of the physique information.

Features of the present disclosure will become apparent from the following description of embodiments with reference to the attached drawings. The following description of embodiments is described by way of example.

### BRIEF DESCRIPTION OF THE DRAWINGS

FIG. 1 is a block diagram illustrating a hardware configuration of an HMD according to one or more aspects of the present disclosure.

FIG. 2 is a block diagram illustrating a functional configuration of the HMD according to one or more aspects of the present disclosure.

FIGS. 3A to 3D are diagrams each illustrating a displayed image according to one or more aspects of the present disclosure.

FIG. 4 is a flowchart illustrating a process according to one or more aspects of the present disclosure.

FIG. 5 is a block diagram illustrating a functional configuration of the HMD according to one or more aspects of the present disclosure.

FIGS. 6A to 6C are diagrams illustrating a method of estimating a compression strength according to one or more aspects of the present disclosure.

FIGS. 7A to 7D are diagrams each illustrating a displayed image according to one or more aspects of the present disclosure.

FIG. 8 is a flowchart illustrating a process according to one or more aspects of the present disclosure.

FIG. 9 is a block diagram illustrating a functional configuration of the HMD according to one or more aspects of the present disclosure.

FIGS. 10A and 10B are diagrams each illustrating a displayed image according to one or more aspects of the present disclosure.

FIG. 11 is a flowchart illustrating a process according to one or more aspects of the present disclosure.

FIG. 12 is a block diagram illustrating a functional configuration of the HMD according to one or more aspects of the present disclosure.

### DESCRIPTION OF THE EMBODIMENTS

### First Embodiment

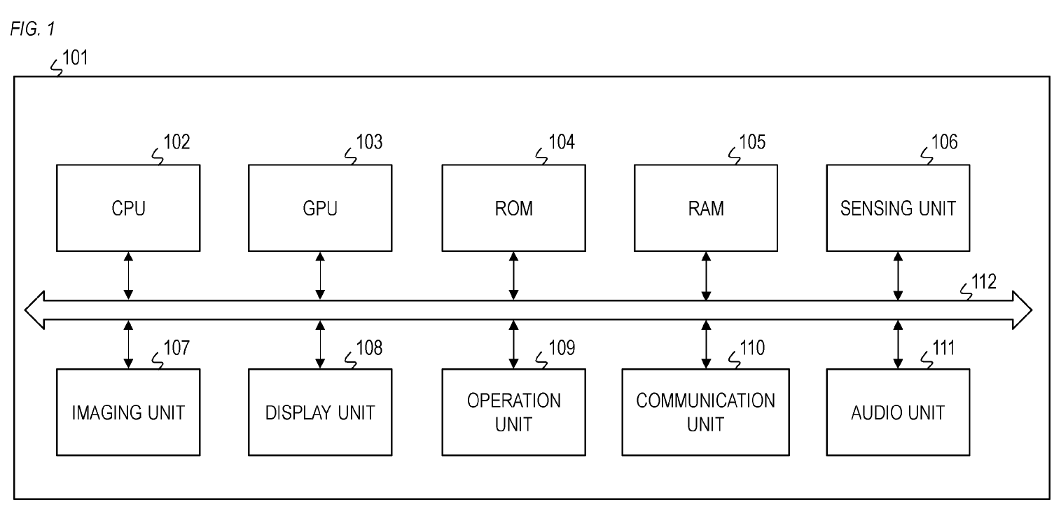

A configuration and an operation of a head-mounted display (HMD) 101, which is an example of electronic device according to a first embodiment, will be described. An operation of the HMD 101 will hereinafter be described that is performed in a case where a user of the HMD 101 provides chest compression treatment to an injured or diseased person (hereinafter, injured/diseased person) in cardiopulmonary arrest. FIG. 1 is a block diagram illustrating a hardware configuration of the HMD 101. The HMD 101 is a video see-through HMD for use by a user. Note that the HMD 101 is not limited to a video see-through HMD, but may also be another type of HMD including, for example, an optical see-through HMD such as a smart glass. Other display apparatus may also be used instead of an HMD. The treatment to be provided by the user is not limited to chest compression treatment, but may also be, for example, massage. The subject of the treatment is not limited to an injured/diseased person. The present disclosure is directed not only to a display apparatus. The present disclosure may also be applied to an information processing apparatus such as a personal computer connected to a display apparatus.

The HMD 101 includes a central processing unit (CPU) 102, a graphics processing unit (GPU) 103, a read-only memory (ROM) 104, a random access memory (RAM) 105, a sensing unit 106, an imaging unit 107, a display unit 108, an operation unit 109, a communication unit 110, and an audio unit 111. These components are connected to each other via a bus 112.

The CPU 102 is an arithmetic processing unit that centrally controls the HMD 101. The CPU 102 executes various programs stored in the ROM 104 and the like to perform various processes.

The GPU 103 is an image processing device that performs rendering, real-time drawing, and the like in computer graphics (CG) according to an instruction from the CPU 102.

The ROM 104 is a nonvolatile read-only memory for storing a program needing no change (e.g., an image processing program), initial data, parameters, and the like.

The RAM 105 temporarily stores input information, calculation results in image processing, and the like. The RAM 105 is a readable and writable volatile memory, and provides the CPU 102 with a work area.

The sensing unit 106 includes a plurality of sensors and the like. The sensing unit 106 may include an inertial measurement unit (IMU) including an acceleration sensor, a gyro sensor, a geomagnetic sensor, and the like. The sensing unit 106 detects a rotation, an inclination, and a movement amount of the head of the user to acquire position and posture information of the head of the user and the like. The sensing unit 106 includes an optical sensor and/or the like. The optical sensor acquires information about the user, such as hand-tracking information regarding a hand of the user and body-tracking information regarding the body of the user. The optical sensor also acquires information regarding the real space, such as point cloud data of a real object disposed in a surrounding real space, depth information of the real object, and position and posture information of the real object.

The imaging unit 107 is a built-in camera or the like of the HMD 101, that converts light into an electrical video signal. The imaging unit 107 includes, for example, an optical system such as an optical lens unit, an imaging element for converting light introduced through the optical lens unit into an electrical video signal, and the like. The imaging unit 107 captures an image of a space in front of the user to acquire a reality image, which is a captured image of the real space. The imaging unit 107 may be, for example, a dual-lens camera (stereo camera) including a camera provided to fit the left eye of the user and a camera provided to fit the right eye of the user. The imaging unit 107 may be capable of acquiring an image having two image areas (stereo image) having a parallax between an image for the right eye and an image for the left eye.

The display unit 108 is a display device such as an organic electroluminescence (EL) display or a liquid crystal display. The display unit 108 displays, to the user, a reality image captured by the imaging unit 107, a CG image of a rendered virtual object or the like, and the like.

The operation unit 109 includes a power button of the HMD 101 and an operational member such as a dial for selecting a mode. In addition, an input interface such as a keyboard and/or a mouse (pointing device) may be connected to the HMD 101.

The communication unit 110 transmits and receives data to and from an external device by wired communication or wireless communication (wireless local area network (LAN), local 5G network, or the like). In the case of the first embodiment, the communication unit 110 may transmit the position and posture information of the HMD 101 (the head of the user) detected by the HMD 101, the position and posture information of the real object, and the like to an external device such as a server via a network. In addition, in a case where an external sensor or the like is connected to the HMD 101, point cloud data of the real object, depth information of the real object, position and posture information of the real object, and the like detected by the external sensor may be received. In a case where an information processing apparatus is connected to the HMD 101, image information of a virtual object rendered by the information processing apparatus may be received.

The audio unit 111 includes a speaker or the like. The audio unit 111 outputs a voice to the user.

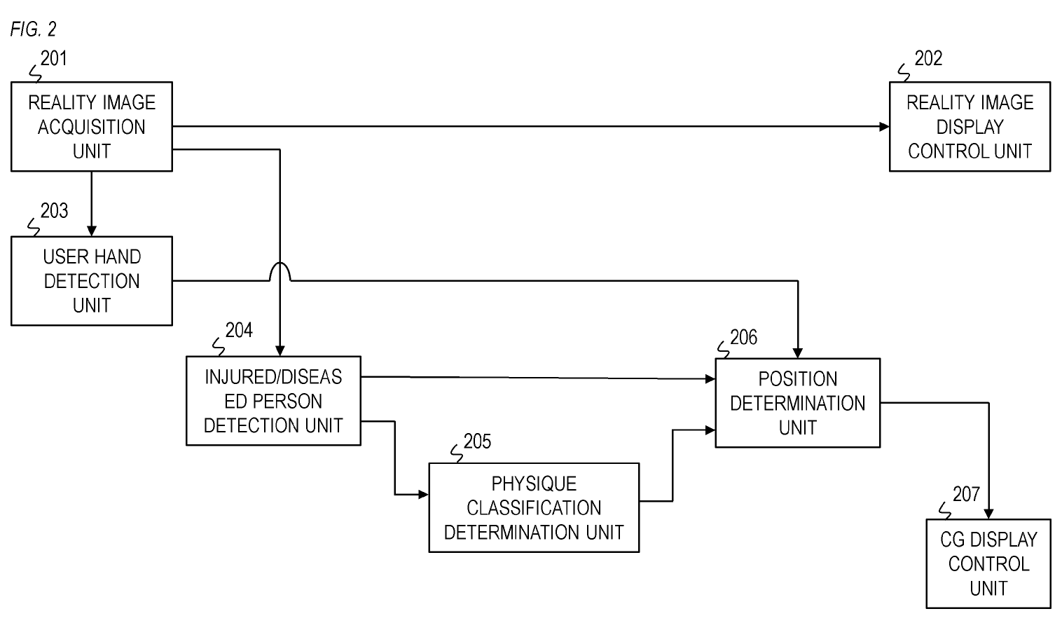

FIG. 2 is a block diagram illustrating a functional configuration of the HMD 101. The HMD 101 includes a reality image acquisition unit 201, a reality image display control unit 202, a user hand detection unit 203, an injured/diseased person detection unit 204, a physique classification determination unit 205, a position determination unit 206, and a CG display control unit 207. In the first embodiment, the functions illustrated in FIG. 2 are implemented by the CPU 102 executing a program. However, the CPU 102 does not need to realize all the functions. For example, the HMD 101 may include a dedicated processing circuit that realizes one or more of the functions illustrated in FIG. 2.

The reality image acquisition unit 201 acquires a reality image captured by the imaging unit 107.

The reality image display control unit 202 performs control to cause the reality image acquired by the reality image acquisition unit 201 to be displayed on the display unit 108.

The user hand detection unit 203 applies an image recognition technique to the reality image acquired by the reality image acquisition unit 201. As a result, a hand of the user is detected. The user hand detection unit 203 acquires, as hand information, the position and posture information of a hand of the user acquired using an image recognition technique. However, the method of acquiring the hand information is not limited to the method of acquiring the hand information from the reality image, but the hand information may be acquired, for example, on the basis of a detection result of the sensing unit 106 or the like.

The injured/diseased person detection unit 204 applies an image recognition technique to the reality image acquired by the reality image acquisition unit 201. As a result, the injured/diseased person is detected, and information regarding the injured/diseased person is acquired. The injured/diseased person detection unit 204 uses an image recognition technique to acquire positional and postural information of the injured/diseased person, acquire age information and the like by estimating the age of the injured/diseased person, and acquire skeletal frame information by estimating the skeletal frame of the injured/diseased person. However, the method of acquiring the information regarding the injured/diseased person is not limited to the method of acquiring such information from the reality image, but such information may be acquired, for example, on the basis of a detection result of the sensing unit 106 or the like.

The physique classification determination unit 205 determines which of three classifications the physique classification (physique information) of the injured/diseased person falls under, where the three classifications are infant, child, and adult, on the basis of the information acquired by the injured/diseased person detection unit 204 (which determination is referred to as physique information acquisition). The determination of the physique classification is made using information regarding physical features such as the age information and the skeletal frame information, included in the information acquired by the injured/diseased person detection unit 204. When the physique classification is determined using the age information, the physique classification determination unit 205 determines a person less than one year of age as an infant, a person from one year of age to less than 16 years of age as a child, and a person of 16 years of age or older as an adult. When the physique classification is determined using the skeletal frame information, the physique classification determination unit 205 determines the physique classification on the basis of the head-to-body ratio, the ratio between the head width and the shoulder width, and/or the like, of the injured/diseased person. When the physique classification is determined on the basis of the head-to-body ratio, the physique classification determination unit 205 determines an injured/diseased person having a head-to-body ratio less than 4 as an infant, an injured/diseased person having a head-to-body ratio equal to or greater than 7 as an adult, and an injured/diseased person having a head-to-body ratio equal to or greater than 4 and less than 7 as a child. A typical standard provides that a person having a head-to-body ratio less than 4 is around 1 year old, and that a person having a head-to-body ratio equal to or greater than 7 is around 16 years old. When the physique classification is determined on the basis of the ratio between the head width and the shoulder width, a determination may be made in such a manner that an injured/diseased person having a shoulder width that is less than 1.5 times the head width is an infant, an injured/diseased person having a shoulder width that is equal to or greater than 2.5 times the head width is an adult, and an injured/diseased person having a shoulder width that is equal to or greater than 1.5 times and less than 2.5 times the head width is a child.

The criteria for determining the physique classification using the age information and the criteria for determining the physique classification using the skeletal frame information are not limited to the criteria described above. In addition, the thresholds for use in determination of the physique classification may be predetermined values preset by a manufacturer, may be values designated by a user, or may be values automatically determined in the HMD 101. The thresholds may be determined in consideration of personal characteristics of the injured/diseased person such as race, gender, and/or past injury or disease history. Determination of the physique classification may be made using only arbitrary one of the foregoing methods or using a combination of a plurality of ones of the foregoing methods. In the first embodiment, the physique classification candidates are the three classifications of infant, child, and adult, but the number of classifications is not limited to three, but may be two or four or more. In the case of the four classifications, the physique classification candidates may include, but do not need to include, all of the three classifications of infant, child, and adult. The physique information is not limited to a physique classification, but may be any information regarding the physique of the injured/diseased person. The age information or the skeletal frame information itself may be used as the physique information.

The position determination unit 206 determines an appropriate compression position (position where chest compression treatment is to be provided) for the injured/diseased person on the basis of the physique classification determined by the physique classification determination unit 205. The position determination unit 206 determines whether the hand of the user detected by the user hand detection unit 203 is at an appropriate compression position. When the hand of the user is not at an appropriate compression position, the position determination unit 206 generates information for presenting a notification. The information for making this notification is information for notifying the user of information such as the direction of deviation of the compression position of the user (the position of the treatment provided by the user) from the appropriate compression position, among upward, downward, leftward, and rightward directions.

The CG display control unit 207 performs control to generate a virtual object on the basis of the appropriate compression position determined by the position determination unit 206. The CG display control unit 207 superimposes the virtual object generated, on the reality image and displays a resulting image on the display unit 108.

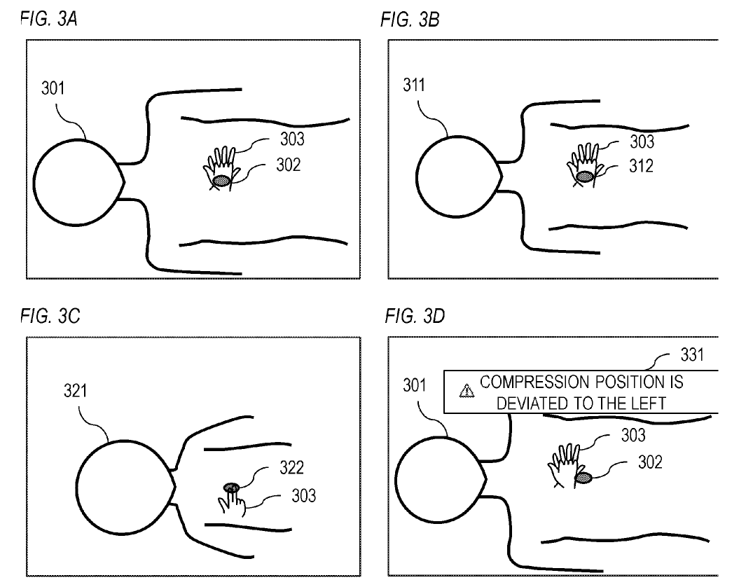

FIGS. 3A to 3D are diagrams for describing an example in which information regarding the appropriate compression position is superimposed and displayed on a reality image on the basis of the physique classification of an injured/diseased person. Each of FIGS. 3A to 3D is a diagram illustrating an image displayed on the display unit 108. The user confirms the information regarding the appropriate compression position by viewing the display unit 108.

FIG. 3A illustrates an example of indicating the appropriate compression position by a display indication in a case where the physique classification of an injured/diseased person is adult. An adult injured/diseased person 301 is an injured/diseased person whose physique classification has been determined to be adult by the physique classification determination unit 205. A virtual object 302 is a virtual object indicating the appropriate compression position on the adult injured/diseased person 301. Regarding adults, in general, the center of a line connecting right and left nipples of the injured/diseased person is said to be the appropriate compression position. The position determination unit 206 detects the positions of the nipples of the adult injured/diseased person 301 using an image recognition technique or the like, and determines the appropriate compression position on the basis of the detected positions of the nipples. The CG display control unit 207 superimposes the virtual object 302 indicating the appropriate compression position on the reality image. A hand 303 represents the hand of the user included in the reality image.

FIG. 3B illustrates an example of indicating the appropriate compression position by a display indication in a case where the physique classification of an injured/diseased person is child. A child injured/diseased person 311 is an injured/diseased person whose physique classification has been determined to be child by the physique classification determination unit 205. A virtual object 312 is a virtual object indicating the appropriate compression position on the child injured/diseased person 311. Regarding children, in general, the center of a line connecting right and left nipples of the injured/diseased person is said to be the appropriate compression position. The position determination unit 206 detects the positions of the nipples of the child injured/diseased person 311 using an image recognition technique or the like, and determines the appropriate compression position on the basis of the detected positions of the nipples. The CG display control unit 207 superimposes the virtual object 312 indicating the appropriate compression position on the reality image.

FIG. 3C illustrates an example of indicating the appropriate compression position by a display indication in a case where the physique classification of an injured/diseased person is infant. An infant injured/diseased person 321 is an injured/diseased person whose physique classification has been determined to be infant by the physique classification determination unit 205. A virtual object 322 is a virtual object indicating the appropriate compression position on the infant injured/diseased person 321. Regarding infants, in general, a position the width of one areola mammae (about 1 to 2 cm) spaced toward the feet from the center of a line connecting left and right nipples of an injured/diseased person is said to be the appropriate compression position. The position determination unit 206 detects the positions of the nipples, the position of the face, and the width of one areola mammae of the infant injured/diseased person 321 using an image recognition technique or the like, and determines the appropriate compression position on the basis of the positions of the nipples, the position of the face, and the width of one areola mammae detected. The CG display control unit 207 superimposes the virtual object 322 indicating the appropriate compression position on the reality image.

FIG. 3D illustrates an example of presenting a notification to guide the user to provide a right treatment when the treatment being provided by the user is different from the treatment that is to be provided. In FIG. 3D, the user is notified that the position of the chest compression being performed by the user is deviated to the left from the position where the compression is to be performed. In FIG. 3D, a compression position warning 331 is displayed, which is a virtual object notifying the user that the position of the hand 303 is deviated to the left from the appropriate compression position. The CG display control unit 207 removes the compression position warning 331 when the position of the hand of the user overlaps the appropriate compression position. FIG. 3D illustrates a case where the physique classification determination unit 205 determines that the physique classification is adult. When the physique classification determination unit 205 determines that the physique classification is child or infant, the CG display control unit 207 also presents, in a similar manner, a notification for guiding the user to provide a right treatment.

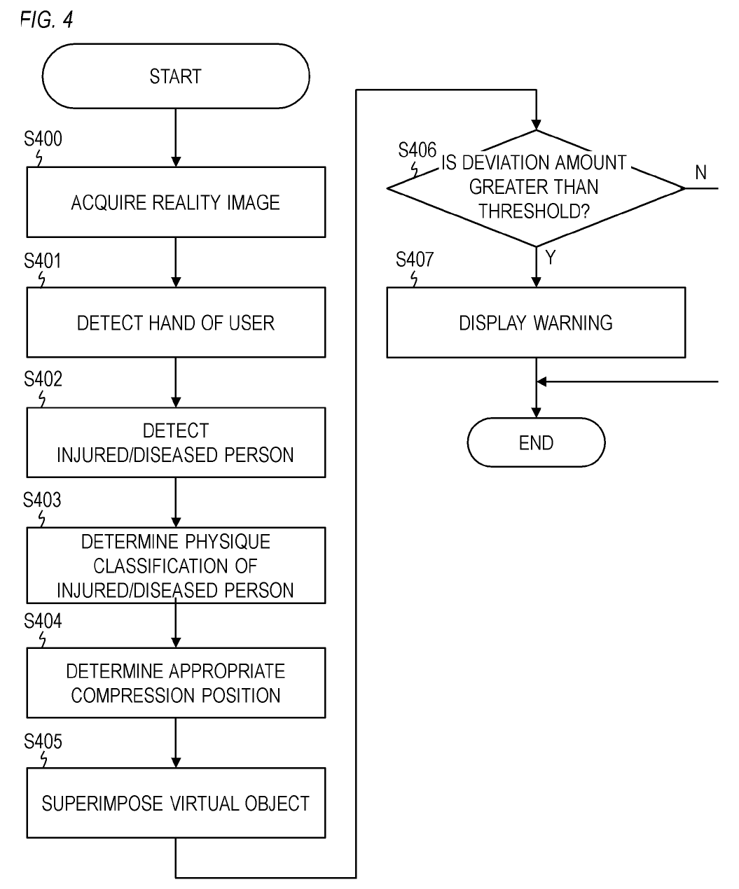

FIG. 4 is a flowchart illustrating a process performed by the CPU 102. A process of displaying an appropriate compression position will be described with reference to the flowchart of FIG. 4. The process of this flowchart may be periodically performed for each frame or with a specific time period.

In S400, the reality image acquisition unit 201 acquires a reality image captured by the imaging unit 107.

In S401, the user hand detection unit 203 detects a hand of the user by applying an image recognition technique to the reality image acquired in S400, and acquires hand information.

In S402, the injured/diseased person detection unit 204 detects the injured/diseased person by applying an image recognition technique to the reality image acquired in S400, and acquires position and posture information, age information, and the like of the injured/diseased person.

In S403, the physique classification determination unit 205 determines the physique classification of the injured/diseased person on the basis of the information acquired in S402.

In S404, the position determination unit 206 determines an appropriate compression position on the basis of the physique classification of the injured/diseased person determined in S403.

In S405, the CG display control unit 207 superimposes and displays the virtual object indicating the appropriate compression position determined in S404, on the reality image. The virtual object is displayed at the appropriate compression position (the position in the reality image corresponding to the appropriate compression position).

In S406, the position determination unit 206 determines whether a deviation amount between the hand information acquired in S401 and the appropriate compression position determined in S404 is greater than a preset threshold. When the deviation amount is greater than the preset threshold, the position determination unit 206 determines that the compression position of the user is not appropriate. The process then proceeds to S407. In addition, when the deviation amount is greater than the threshold, the position determination unit 206 estimates the direction and the amount of the deviation of the hand of the user from the appropriate compression position. When the deviation amount is equal to or less than the preset threshold, the position determination unit 206 determines that the compression position of the user is appropriate. The process of FIG. 4 then terminates.

In S407, the CG display control unit 207 superimposes the compression position warning 331 on the reality image as illustrated in FIG. 3D. The process of FIG. 4 then terminates.

As described above, according to the first embodiment, treatment for an injured/diseased person can be assisted by a notification to the user who provides the treatment, of the position where chest compression treatment is to be provided. According to the first embodiment, the position where the treatment is to be provided can be visually indicated, thereby enabling the treatment for the injured/diseased person provided by the user to be suitably assisted even when noise makes an instruction scarcely audible. Note that, although an example has been described in which a notification is presented by a display indication, the notification method is not limited thereto. The notification may be made by, for example, a voice output.

### Second Embodiment

The first embodiment has been described in which information regarding the appropriate compression position is superimposed and displayed on a reality image, as the information for assisting the treatment. A second embodiment will next be described in which information regarding an appropriate compression strength (i.e., strength to be provided in the chest compression treatment) is superimposed and displayed on a reality image, as the information for assisting the treatment.

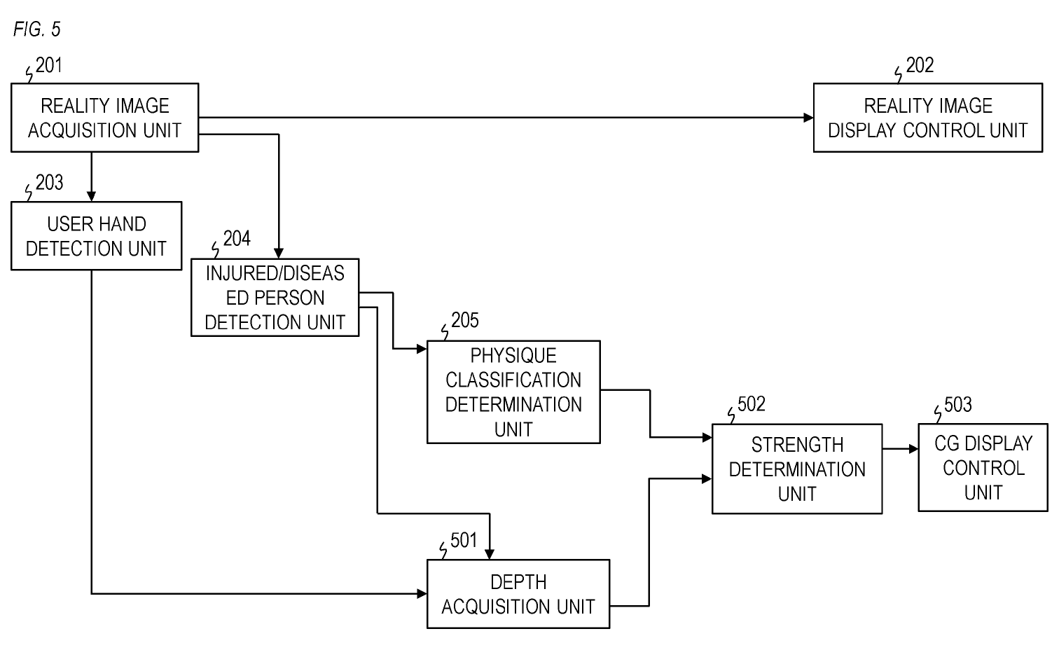

FIG. 5 is a block diagram illustrating a functional configuration of the HMD 101 according to the second embodiment. The reality image acquisition unit 201, the reality image display control unit 202, the user hand detection unit 203, the injured/diseased person detection unit 204, and the physique classification determination unit 205 are similar to those of FIG. 2, and description thereof will therefore be omitted. In the second embodiment, the functions illustrated in FIG. 5 are implemented by the CPU 102 executing a program. However, the CPU 102 does not need to realize all the functions. For example, the HMD 101 may include a dedicated processing circuit that realizes one or more of the functions illustrated in FIG. 5.

A depth acquisition unit 501 acquires point cloud data and depth information such as a depth map, regarding a hand of the user and the injured/diseased person, which are real objects, from the detection result of the sensing unit 106 and/or the like. When the image acquired by the imaging unit 107 is a stereo image, the depth information may be calculated using parallax between two image areas thereof.

A strength determination unit 502 determines an appropriate compression strength on the basis of the physique classification of the injured/diseased person determined by the physique classification determination unit 205. For example, the strength determination unit 502 uses a predetermined table that presents a correspondence relationship between the physique classification and an appropriate compression strength to determine the appropriate compression strength corresponding to the physique classification determined by the physique classification determination unit 205. The table presenting the correspondence relationship between the physique classification and the appropriate compression strength may be provided singly or plurally. For example, a plurality of tables may be provided that take into consideration personal characteristics such as race, gender, and past injury or disease history. When a plurality of tables are provided, a table to be used is manually or automatically selected from the plurality of tables. When one table is provided, the table may be adjusted manually or automatically in consideration of personal characteristics and the like.

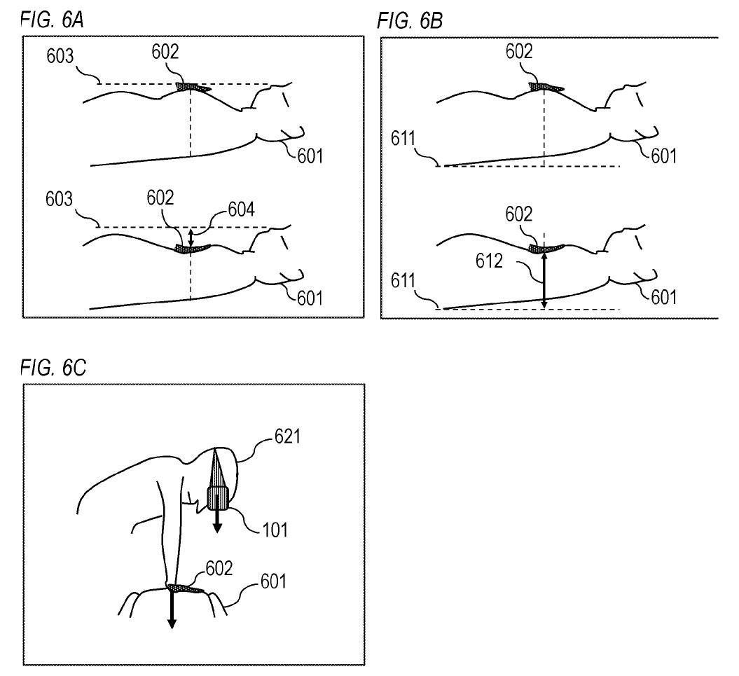

The strength determination unit 502 estimates the compression strength provided by the user (hereinafter, the compression strength of the user, as appropriate), on the basis of the point cloud data, the depth information, and the like regarding a hand of the user and the injured/diseased person, acquired by the depth acquisition unit 501. A method of estimating the compression strength of the user will next be described with reference to FIGS. 6A to 6C.

In the method illustrated in FIG. 6A, the strength determination unit 502 measures a depth 604 of a hand 602 of the user with respect to a height 603 of the nose of an injured/diseased person 601 (i.e., the distance from the nose of the injured/diseased person 601 to the hand 602 of the user) on the basis of the information acquired by the depth acquisition unit 501. The depth 604 is measured with the direction toward the ground defined as the positive direction. The strength determination unit 502 makes an estimation in such a manner that a greater depth 604 indicates a stronger compression strength.

In the method illustrated in FIG. 6B, the strength determination unit 502 measures a depth 612 of the hand 602 of the user with respect to a height 611 of the ground (i.e., the distance from the ground to the hand 602 of the user) on the basis of the information acquired by the depth acquisition unit 501. The strength determination unit 502 makes an estimation in such a manner that a smaller depth 612 indicates a stronger compression strength.

In FIG. 6C, a user 621 wearing the HMD 101 performs chest compression on the injured/diseased person 601. When the injured/diseased person is an adult or a child, the position of the hand 602 of the user and the position of the head of the user 621 move in the vertical direction during chest compression. Thus, in the method illustrated in FIG. 6C, the position and posture information of the head of the user 621 is acquired from the detection result of the sensing unit 106, and an estimation is made in such a manner that a larger amount of change in the position of the head from the position when the head of the user 621 is at the highest position indicates a stronger compression strength.

When the injured/diseased person is an infant or in another similar case, there may be a case where chest compression is performed in which the head of the user moves very little and only a hand of the user moves vertically. In such a case, estimation may be made in such a manner that a larger amount of change in the position of the hand from the position when the hand of the user acquired by the user hand detection unit 203 is at the highest position indicates a stronger compression strength. Estimation of the compression strength may be made using a combination of the plurality of methods described above, made by allowing a selection of any method depending on the physique classification, or made using a method preset by the manufacturer depending on the physique classification.

In addition, the strength determination unit 502 determines whether the compression strength provided by the user is appropriate. When the compression strength provided by the user is not the appropriate compression strength, the strength determination unit 502 generates information for presenting a notification. The information for making this notification is information for notifying the user that the compression strength provided by the user is greater or less than the appropriate compression strength.

A CG display control unit 503 performs control to generate a virtual object on the basis of the appropriate compression strength determined by the strength determination unit 502 and the compression strength of the user. The CG display control unit 503 superimposes the virtual object generated, on the reality image and displays a resulting image on the display unit 108.

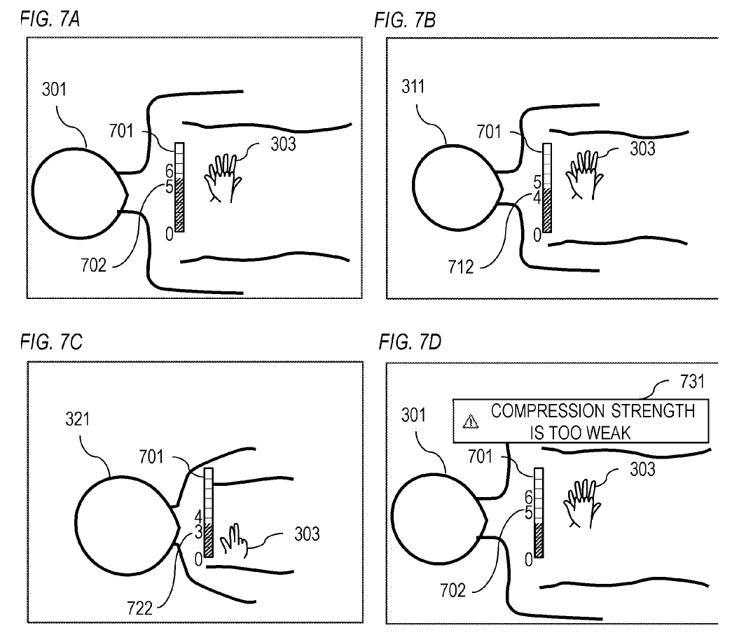

FIGS. 7A to 7D are diagrams for describing an example in which information regarding the appropriate compression strength is superimposed and displayed on a reality image on the basis of the physique classification of an injured/diseased person. The following description describes the compression strength as a depression amount (cm) of the breastbone of the injured/diseased person. Each of FIGS. 7A to 7D is a diagram illustrating an image displayed on the display unit 108. The user confirms the information regarding the appropriate compression strength by viewing the display unit 108.

FIG. 7A illustrates an example of indicating the appropriate compression strength by a display indication in a case where the physique classification of an injured/diseased person is adult. A virtual object 701 is a virtual object indicating the compression strength of the user (the strength provided in the treatment by the user). For example, the virtual object 701 is a virtual object indicating the level by stages, such as a virtual object of a level meter, and indicates the compression strength of the user in real time. A strength indicator 702 is a virtual object indicating the appropriate compression strength. Regarding adults, in general, chest compression is preferably performed with a strength to cause a 5 to 6-cm depression. The strength indicator 702 accordingly indicates a strength to cause a 5 to 6-cm depression as the appropriate compression strength.

FIG. 7B illustrates an example of indicating the appropriate compression strength by a display indication in a case where the physique classification of an injured/diseased person is child. A strength indicator 712 is a virtual object indicating the appropriate compression strength. Regarding children, in general, chest compression is preferably performed with a strength to cause about one third of the thickness of the breast or a 4 to 5-cm depression. The strength indicator 712 accordingly indicates a strength to cause a 4 to 5-cm depression as the appropriate compression strength.

FIG. 7C illustrates an example of indicating the appropriate compression strength by a display indication in a case where the physique classification of an injured/diseased person is infant. A strength indicator 722 is a virtual object indicating the appropriate compression strength. Regarding infants, in general, chest compression is preferably performed with a strength to cause about one third of the thickness of the breast or a 3 to 4-cm depression. The strength indicator 722 accordingly indicates a strength to cause a 3 to 4-cm depression as the appropriate compression strength.

FIG. 7D illustrates an example of presenting a notification to guide the user to provide a right treatment when the treatment being provided by the user is different from the treatment that is to be provided. In FIG. 7D, the user is notified that the strength of the chest compression being performed by the user is less than the compression strength to be provided. In FIG. 7D, a compression strength warning 731 is displayed, which is a virtual object notifying the user that the compression strength of the user is less than the appropriate compression strength. The compression strength warning 731 notifies the user that the compression strength is strong when the compression strength of the user is greater than the appropriate compression strength. The CG display control unit 503 removes the compression strength warning 731 when the compression strength of the user becomes the appropriate compression strength. FIG. 7D illustrates a case where the physique classification determination unit 205 determines that the physique classification is adult. When the physique classification determination unit 205 determines that the physique classification is child or infant, the CG display control unit 503 also presents, in a similar manner, a notification for guiding the user to provide a right treatment.

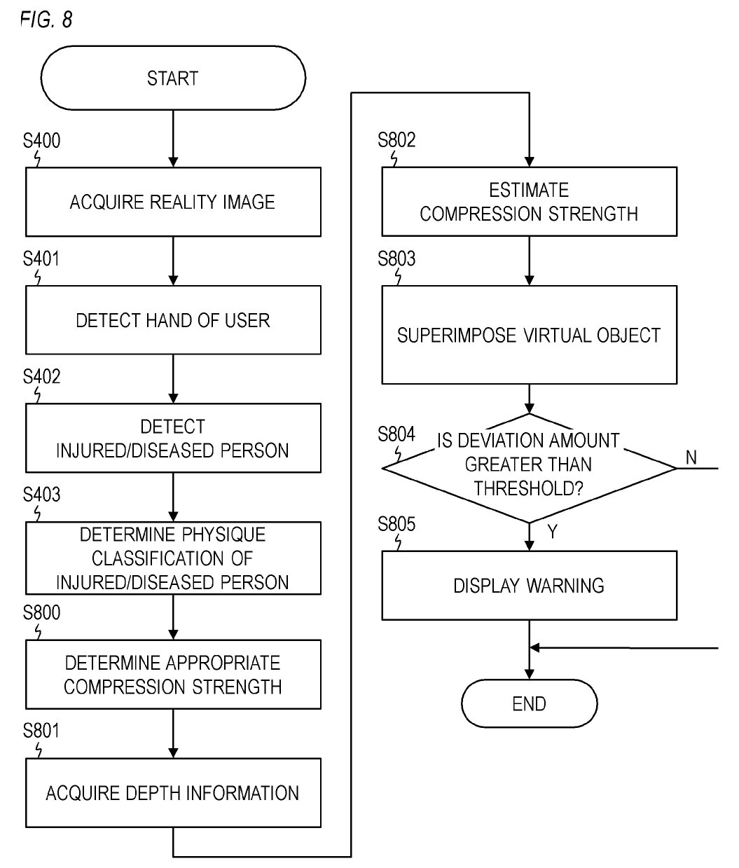

FIG. 8 is a flowchart illustrating a process performed by the CPU 102. A process of displaying an appropriate compression strength will be described with reference to the flowchart of FIG. 8. The process of this flowchart may be periodically performed for each frame or with a specific time period. In the description of FIG. 8, only steps different from the steps of the flowchart of FIG. 4 will be described.

In S800, the strength determination unit 502 determines the appropriate compression strength on the basis of the physique classification of the injured/diseased person determined in S403.

In S801, the depth acquisition unit 501 acquires point cloud data and depth information regarding the hand of the user and the injured/diseased person.

In S802, the strength determination unit 502 estimates the compression strength provided by the user, on the basis of the point cloud data, the depth information, and the like regarding the hand of the user and the injured/diseased person, acquired in S801.

In S803, the CG display control unit 503 superimposes and displays the virtual object indicating the appropriate compression strength determined in S800 and the virtual object indicating the compression strength provided by the user estimated in S802, on the reality image.

In S804, the strength determination unit 502 determines whether a deviation amount (absolute value) between the appropriate compression strength determined in S800 and the current compression strength of the user estimated in S802 is greater than a preset threshold. When the deviation amount is greater than the preset threshold, the strength determination unit 502 determines that the compression strength of the user is not appropriate. The process then proceeds to S805. In addition, when the deviation amount is greater than the threshold, the strength determination unit 502 estimates whether the compression strength of the user is greater or less than the appropriate compression strength. When the deviation amount is equal to or less than the preset threshold, the strength determination unit 502 determines that the compression strength of the user is appropriate. The process of FIG. 8 then terminates.

In S805, the CG display control unit 503 superimposes the compression strength warning 731 on the reality image as illustrated in FIG. 7D. The process of FIG. 8 then terminates.

As described above, according to the second embodiment, treatment provided by the user can be assisted by a notification to the user who provides the treatment for an injured/diseased person, of the strength to be provided in chest compression treatment. According to the second embodiment, the strength to be provided in the treatment can be visually indicated, thereby enabling the treatment for the injured/diseased person provided by the user to be suitably assisted even when noise makes an instruction scarcely audible.

### Third Embodiment

The first embodiment has been described in which information regarding the appropriate compression position is superimposed and displayed on a reality image, as the information for assisting the treatment. The second embodiment has been described in which information regarding the appropriate compression strength is superimposed and displayed on a reality image, as the information for assisting the treatment. A third embodiment will next be described in which information regarding an appropriate compression rate (i.e., rate of the chest compression treatment to be provided) is superimposed and displayed on a reality image, as the information for assisting the treatment.

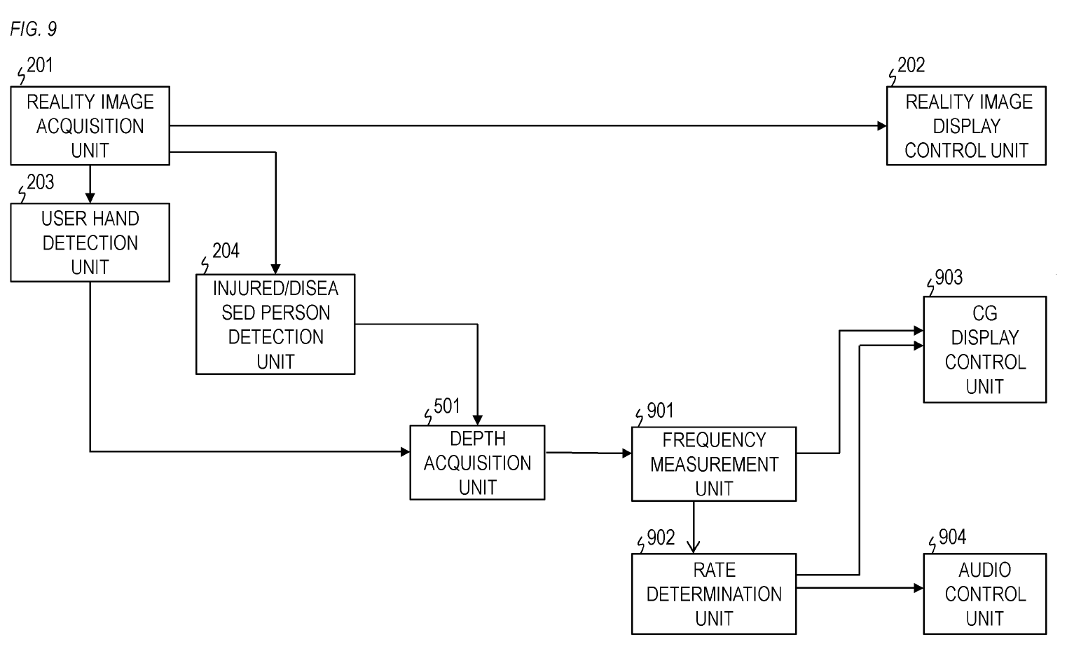

FIG. 9 is a block diagram illustrating a functional configuration of the HMD 101 according to the third embodiment. The reality image acquisition unit 201, the reality image display control unit 202, the user hand detection unit 203, and the injured/diseased person detection unit 204 are similar to those of FIG. 2, and description thereof will therefore be omitted. The depth acquisition unit 501 is similar to that of FIG. 5, and description thereof is therefore omitted. In the third embodiment, the functions illustrated in FIG. 9 are implemented by the CPU 102 executing a program. However, the CPU 102 does not need to realize all the functions. For example, the HMD 101 may include a dedicated processing circuit that realizes one or more of the functions illustrated in FIG. 9.

A frequency measurement unit 901 measures the number of times of consecutively performed chest compression (the number of times of consecutive compression) performed by the user, using point cloud data and depth information such as a depth map, regarding a hand of the user and the injured/diseased person, and the like, acquired by the depth acquisition unit 501. The method for measuring the number of times of consecutive compression is not particularly limited. For example, the method may be such that a measurement is made of the depth of a hand of the user (or of the breastbone of the injured/diseased person) with respect to the height of the chin or the height of the nose of the injured/diseased person or the height of the ground, and the number of times the depth exceeds or falls below a preset threshold is counted as the number of times of consecutive compression. Alternatively, the method may be such that the number of times of exceeding or falling below a preset threshold by a change amount of the position of the hand of the user from the position when the hand detected by the user hand detection unit 203 is at the highest position is counted as the number of times of consecutive compression. Still alternatively, the method may be such that the position and posture information of the head of the user is acquired from the detection result of the sensing unit 106, and the number of times of exceeding or falling below a preset threshold by a change amount of the position of the head of the user from the position when the head of the user is at the highest position is counted as the number of times of consecutive compression.

A rate determination unit 902 determines an appropriate compression rate (appropriate beats per minute (BPM) value) for the injured/diseased person. The appropriate BPM may be a value preset by the manufacturer or an arbitrary value set by the user. The rate determination unit 902 calculates the current compression rate of the user from an increase rate of the number of times of consecutive compression, and determines whether the compression rate is appropriate. A method for calculating the current compression rate of the user is such that, for example, when the number of times of consecutive compression is increasing by one count in 0.6 seconds, the compression rate of the user can be calculated as 100 BPM.

A CG display control unit 903 performs control to generate a virtual object on the basis of the appropriate compression rate determined by the rate determination unit 902 and the number of times of consecutive compression of the user. The CG display control unit 903 superimposes the virtual object generated, on the reality image and displays a resulting image on the display unit 108.

An audio control unit 904 performs control to output, from the audio unit 111, a sound at the tempo, or to the rhythm, of the appropriate compression rate determined by the rate determination unit 902.

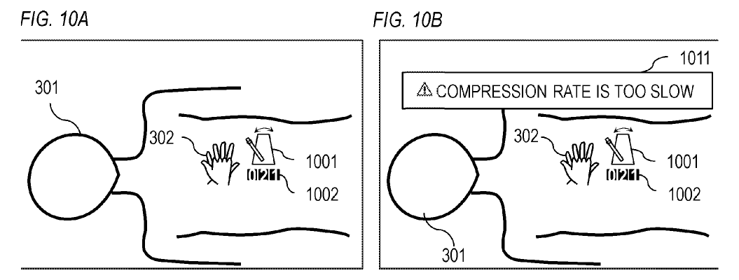

FIGS. 10A and 10B are diagrams for describing an example in which the appropriate compression rate is superimposed and displayed on a reality image. Each of FIGS. 10A and 10B is a diagram illustrating an image displayed on the display unit 108. The user confirms the information regarding the appropriate compression rate by viewing the display unit 108.

FIG. 10A illustrates an example of indicating the appropriate compression rate by a display indication. This example assumes that the injured/diseased person is the adult injured/diseased person 301, but similar description applies to a case where the injured/diseased person is a child or an infant. A virtual object 1001 is a virtual object indicating the appropriate compression rate. The virtual object 1001 is represented in a display form capable of indicating a tempo or rhythm, such as, for example, a metronome, and presents the appropriate compression rate for the user. In general, an appropriate compression rate is 100 to 120 BPM. Therefore, the virtual object 1001 indicates 100 to 120 BPM as the appropriate compression rate. A virtual object 1002 is a virtual object indicating the number of times of consecutive compression. Chest compression is thought to be desirably performed in a combination of 30 consecutive compressions and two artificial breaths. Therefore, the virtual object 1002 indicates the number of times of consecutive compression to prompt the user to perform 30 consecutive compressions and two artificial breaths. The virtual object 1002 may provide an indication that counts up to 30 or counts down from 30.

The display form of the virtual object 1001 is not limited to the metronome form. For example, the display form may be a form such as a bar form that extends and shortens vertically or horizontally in accordance with the tempo or the like, a form in which a predetermined virtual object or at least part of the screen changes color in accordance with the tempo or the like, or a form in which a waveform is displayed in accordance with the tempo or the like.

FIG. 10B illustrates an example of presenting a notification to guide the user to provide a right treatment when the treatment being provided by the user is different from the treatment that is to be provided. FIG. 10B provides an indication to the user by a display indication that the compression rate being provided by the user is lower than the appropriate compression rate. FIG. 10B includes a compression rate warning 1011, which is a virtual object notifying the user that the compression rate of the user is less than the appropriate compression rate. The CG display control unit 903 removes the compression rate warning 1011 when the compression rate provided by the user becomes the appropriate compression rate. FIG. 10B illustrates a case where the injured/diseased person is an adult. When the injured/diseased person is a child or an infant, the CG display control unit 903 also presents, in a similar manner, a notification for guiding the user to provide a right treatment.

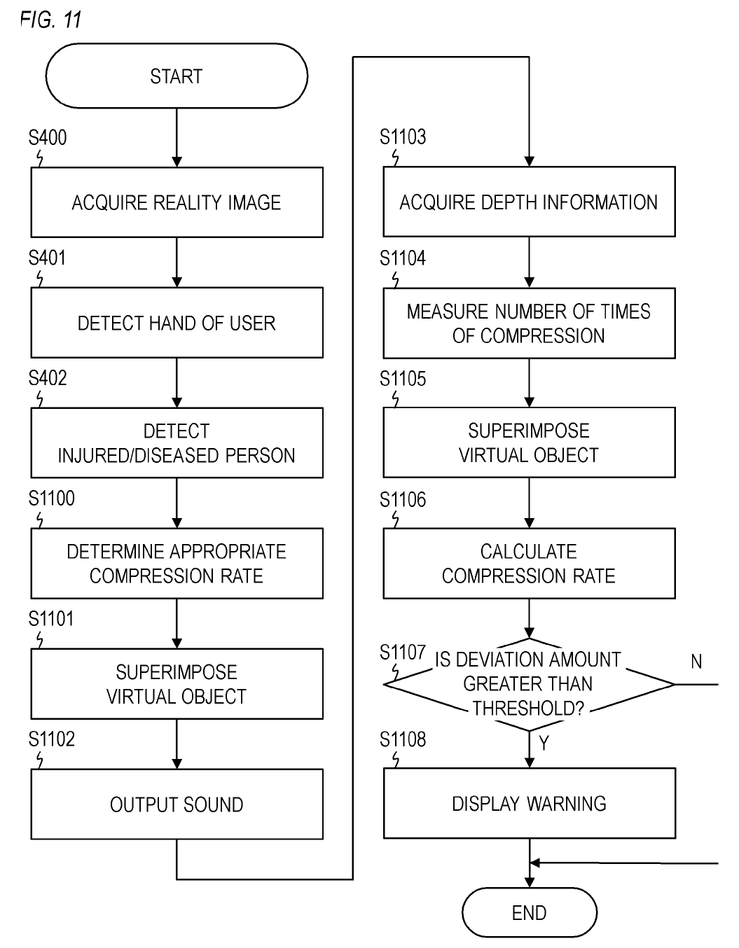

FIG. 11 is a flowchart illustrating a process performed by the CPU 102. A process of displaying an appropriate compression rate will be described with reference to the flowchart of FIG. 11. The process of this flowchart may be periodically performed for each frame or with a specific time period. In the description of FIG. 11, only steps different from the steps of the flowchart of FIG. 4 will be described.

In S1100, the rate determination unit 902 determines an appropriate compression rate.

In S1101, the CG display control unit 903 superimposes and displays the virtual object indicating the appropriate compression rate determined in S1100, on the reality image.

In S1102, the audio control unit 904 outputs a sound at the tempo, or to the rhythm, according to the appropriate compression rate determined in S1100.

In S1103, the depth acquisition unit 501 acquires point cloud data and depth information regarding the hand of the user and the injured/diseased person.

In S1104, the frequency measurement unit 901 measures the number of times of consecutive compression on the basis of the depth information acquired in S1103 and/or the like.

In S1105, the CG display control unit 903 superimposes and displays the virtual object indicating the number of times of consecutive compression determined in S1104, on the reality image.

In S1106, the rate determination unit 902 calculates the compression rate of the user from the increase rate of the number of times of consecutive compression of the user measured in S1104.

In S1107, the rate determination unit 902 determines whether a deviation amount (absolute value) between the appropriate compression rate determined in S1100 and the compression rate of the user calculated in S1106 is greater than a preset threshold. When the deviation amount is greater than the preset threshold, the rate determination unit 902 determines that the compression rate of the user is not appropriate, and causes the process to proceed to S1108. In addition, when the deviation amount is greater than the threshold, the rate determination unit 902 determines whether the compression rate of the user is greater or less than the appropriate compression rate. When the deviation amount is equal to or less than the preset threshold, the process is terminated.

In S1108, the CG display control unit 903 superimposes and displays the compression rate warning 1011 on the reality image as in the example illustrated in FIG. 10B, and terminates the process. The CG display control unit 903 notifies the user that the compression rate is high using the compression rate warning 1011 when the compression rate of the user is higher than the appropriate compression rate. When the compression rate of the user is equal to or less than the appropriate compression rate, the user is notified that the compression rate is low.

As described above, according to the third embodiment, treatment provided by the user can be assisted by a visual and audible notification to the user, of the rate of chest compression treatment to be provided.

### Fourth Embodiment

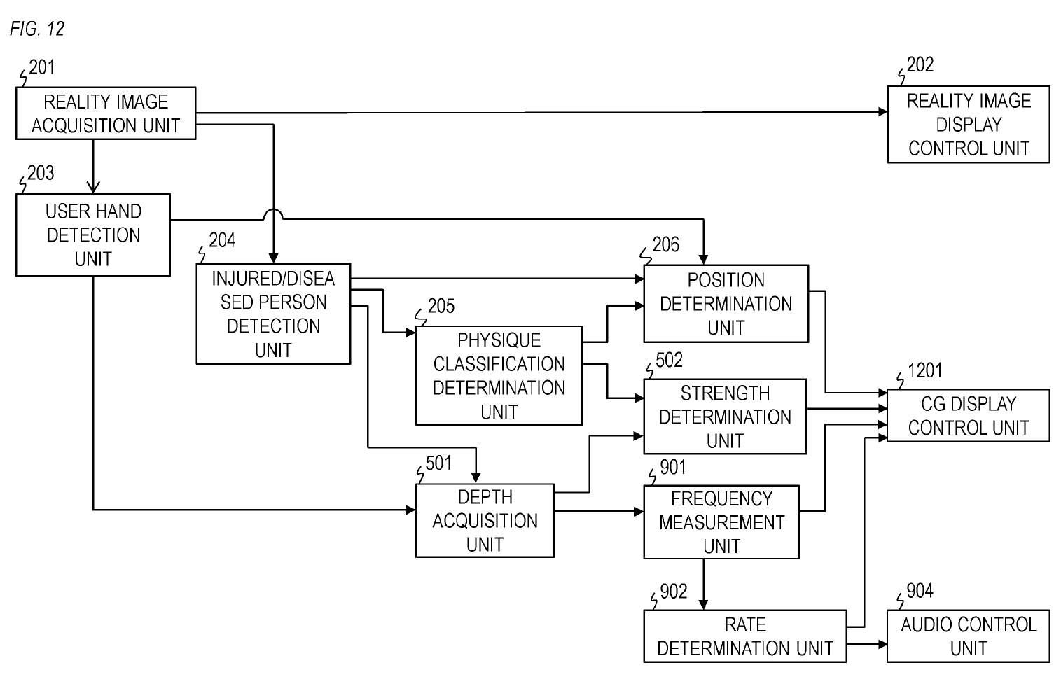

A fourth embodiment is a combination of the first through third embodiments. FIG. 12 is a block diagram illustrating a functional configuration of the HMD 101 according to the fourth embodiment. The reality image acquisition unit 201, the reality image display control unit 202, the user hand detection unit 203, the injured/diseased person detection unit 204, the physique classification determination unit 205, and the position determination unit 206 are similar to those of FIG. 2, and description thereof will therefore be omitted. The depth acquisition unit 501 and the strength determination unit 502 are similar to those of FIG. 5, and description thereof is therefore omitted. The frequency measurement unit 901, the rate determination unit 902, and the audio control unit 904 are similar to those of FIG. 9, and description thereof is therefore omitted. In the fourth embodiment, the functions illustrated in FIG. 12 are implemented by the CPU 102 executing a program.

A CG display control unit 1201 performs control to generate a virtual object on the basis of the appropriate compression position determined by the position determination unit 206. The CG display control unit 1201 also performs control to generate a virtual object on the basis of the appropriate compression strength determined by the strength determination unit 502 and the compression strength of the user. The CG display control unit 1201 further performs control to generate a virtual object on the basis of the appropriate compression rate determined by the rate determination unit 902 and the number of times of consecutive compression of the user measured by the frequency measurement unit 901. The CG display control unit 1201 superimposes the virtual objects generated, on the reality image and displays a resulting image on the display unit 108.

Note that the above-described various types of control may be processing that is carried out by one piece of hardware (e.g., processor or circuit), or otherwise. Processing may be shared among a plurality of pieces of hardware (e.g., a plurality of processors, a plurality of circuits, or a combination of one or more processors and one or more circuits), thereby carrying out the control of the entire device.

Also, the above processor is a processor in the broad sense, and includes general-purpose processors and dedicated processors. Examples of general-purpose processors include a central processing unit (CPU), a micro processing unit (MPU), a digital signal processor (DSP), and so forth. Examples of dedicated processors include a graphics processing unit (GPU), an application-specific integrated circuit (ASIC), a programmable logic device (PLD), and so forth. Examples of PLDs include a field-programmable gate array (FPGA), a complex programmable logic device (CPLD), and so forth.

The embodiment described above (including variation examples) is merely an example. Any configurations obtained by suitably modifying or changing some configurations of the embodiment within the scope of the subject matter of the present disclosure are also included in the present disclosure. The present disclosure also includes other configurations obtained by suitably combining various features of the embodiment.

The present disclosure enables a user who provides a treatment to provide an appropriate treatment regardless of a subject of the treatment.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a 'non-transitory computer-readable storage medium') to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)^{TM}), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-067649, filed Apr. 16, 2025, which is hereby incorporated by reference herein in its entirety.

What is claimed is:

1. An electronic device comprising
one or more processors and memory configured to:
execute an image acquisition process of acquiring a captured image;
execute an information acquisition process of acquiring, from the captured image, physique information regarding a physique of a subject of treatment; and
execute a control process of performing control to provide a user who provides the treatment for the subject, with a notification for assisting the treatment, on a basis of the physique information.

2. The electronic device according to claim 1, wherein
in the information acquisition process, information indicating which of a plurality of classifications the subject falls under is acquired as the physique information, the classifications including at least infant, child, and adult.

3. The electronic device according to claim 1, wherein
in the information acquisition process, an age of the subject is estimated from the captured image, and the physique information is acquired on a basis of the estimated age.

4. The electronic device according to claim 1, wherein
in the information acquisition process, a skeletal frame of the subject is estimated from the captured image, and the physique information is acquired on a basis of the estimated skeletal frame.

5. The electronic device according to claim 1, wherein
in the control process, control is performed to provide a notification of a position where the treatment is to be provided.

6. The electronic device according to claim 1, wherein
in the control process, control is performed to provide a notification of a strength to be provided in the treatment.

7. The electronic device according to claim 6, wherein
in the control process, control is performed to further provide a notification of a strength provided in the treatment by the user.

8. The electronic device according to claim 6, wherein
in the control process, a strength provided in the treatment by the user is acquired from the captured image.

9. The electronic device according to claim 1, wherein
in the control process, control is performed to provide a notification of a number of times of the treatment to be provided.

10. The electronic device according to claim 9, wherein
in the control process , a number of times of the treatment provided by the user is acquired from the captured image.

11. The electronic device according to claim 1, wherein
in the control process, control is performed to provide a second notification in a case where treatment being provided by the user is different from treatment to be provided.

12. The electronic device according to claim 1, wherein
in the control process, control is performed to provide the notification by a display indication.

13. A control method of an electronic device, comprising:
acquiring a captured image;
acquiring, from the captured image, physique information regarding a physique of a subject of treatment; and
performing control to provide a user who provides the treatment for the subject, with a notification for assisting the treatment, on a basis of the physique information.

14. A non-transitory computer readable medium that stores instructions, wherein the instructions causes a computer to execute a control method of an electronic device, the control method comprising:
acquiring a captured image;
acquiring, from the captured image, physique information regarding a physique of a subject of treatment; and
performing control to provide a user who provides the treatment for the subject, with a notification for assisting the treatment, on a basis of the physique information.
